(19) United States
(12) Patent Application Publication
(10) Pub. No.: US 2017/0106052 A1
(43) Pub. Date: Apr. 20, 2017

(54) Insulin Dosage Proposal System

(71) Applicants: Joanneum Research Forschungsgesellschaft MBH, Graz (AT); Medizinische Universität Graz, Graz (AT)

(72) Inventors: Stephan Spat, Deutschlandsberg (AT); Bernhard Höll, Graz (AT); Peter Beck, Hart bei Graz (AT); Thomas Truskaller, Hart bei Graz (AT); Reinhard Moser, Voitsberg (AT); Klaus Donsa, Keutschach am See (AT); Johannes Plank, Graz (AT); Julia Mader, Graz (AT); Katharina Lichtenegger, Eggersdorf (AT); Lukas Schaupp, Graz (AT); Thomas Pieber, Graz (AT)

(21) Appl. No.: 15/308,961

(22) PCT Filed: May 5, 2015
(86) PCT No.: PCT/EP2015/059880

§ 371 Date: Nov. 4, 2016

(30) Foreign Application Priority Data
May 5, 2014 (GB) 1407896.8

(51) Int. Cl. A61K 38/28; A61B 5/00; A61B 5/145; G06F 19/00; A61K 9/00

(52) U.S. Cl. CPC A61K 38/28; G06F 19/3468; G06F 19/327; G06F 19/322; G06F 19/345; A61K 9/0019; A61B 5/14532; A61B 5/4839; A61B 5/742

(57) ABSTRACT

A device for proposing an insulin dosage for a diabetes patient, wherein the device comprises an input interface configured for receiving patient glucose level data indicative of glucose level information of the patient, a processor configured for determining the insulin dosage in an interactive time-dependent manner based on applying the received patient glucose level data to at least only predefined insulin dosage determining criterion, and an output interface configured for outputting a result of the determining as a proposal for the insulin dosage indicative of doses and assigned times of the day according to which insulin is to be administered to the diabetes patient.

### CROSS-REFERENCE TO RELATED APPLICATIONS

This application is a U.S. national phase patent application of PCT/EP2015/059880 which claims the benefit of the filing date of Great Britain Patent Application No. 1407896.8, filed on May 5, 2014, the disclosures of which are hereby incorporated herein by reference in their entirety.

### TECHNICAL FIELD

Embodiments of the invention relate to a device for proposing an insulin dosage for a diabetes patient.

Embodiments of the invention also relate to an arrangement for managing insulin delivery to a plurality of diabetes patients of a medical facility.

Moreover, embodiments of the invention relate to a method of proposing an insulin dosage for a diabetes patient.

Beyond this, embodiments of the invention relate to a program element.

Furthermore, embodiments of the invention relate to a computer-readable medium.

Technological Background

Hyperglycemia in hospitalised patients with diabetes type 2 is a common and costly health care problem with profound medical consequences.

Increasing evidence indicates that the development of hyper glycaemia during acute medical or surgical illness is not a physiologic or benign condition but is a marker of poor clinical outcome and mortality. Observational studies in diabetic subjects admitted to general medical and surgical areas have shown that poor glycaemic control is associated with prolonged hospital stay, infection, disability after hospital discharge and death.

### SUMMARY

There may be a need for a safe and efficient way of managing insulin delivery for diabetes patients.

According to exemplary embodiments, a device for proposing an insulin dosage for a diabetes patient, an arrangement for managing insulin delivery to a plurality of diabetes patients of a medical facility, a method of proposing an insulin dosage for a diabetes patient, a program element, and a computer-readable medium according to the independent claims are provided.

According to an exemplary embodiment of the invention, a device for proposing an insulin dosage for a diabetes patient is provided, wherein the device comprises an input interface (such as a touchscreen, a keypad, a voice recognition system and/or a data line) configured for receiving patient glucose level data indicative of glucose level information of the patient (in particular providing the result of one or more glucose level measurements performed with the patient), a processor configured for determining the insulin dosage in an interactive time-dependent manner based on applying the received patient glucose level data to at least only predefined insulin dosage determining criterion (in particular a criterion concerning when and in which amount which kind of insulin should be administered to the patient in view of one or more measured glucose levels of the patient), and an output interface (such as a display, a microphone, etc.) configured for outputting a result of the determining as a proposal for the insulin dosage indicative of doses (or amounts) and assigned times of the day (in particular indicative of a temporal sequence of insulin administration events) according to which insulin is to be administered to the diabetes patient.

According to another exemplary embodiment of the invention, an arrangement (for instance a network of communicatively coupled nodes in form of devices and control entity) for managing insulin delivery to a plurality of diabetes patients of a medical facility (such as a hospital) is provided, wherein the arrangement comprises a plurality of devices having the above mentioned features, each for proposing an insulin dosage according to which insulin is to be administered to at least a part of the diabetes patients (in particular, each of the devices may be programmed to monitor insulin supply and glucose levels of one or more assigned diabetes patients of the medical facility), and a control entity (in particular a central control computer such as a server of a server-client network in which the devices are implemented as clients) communicatively coupled to the plurality of devices for communicating device control data (such as control commands for controlling operation of the respective device and/or software code to be executed by the respective device) and patient-related data (such as medical and/or personal information concerning the patient which data is used for proposing the insulin dosage) between the control entity and the devices.

According to yet another exemplary embodiment of the invention, a method (for instance a computer-implemented method) of proposing an insulin dosage for a diabetes patient is provided, wherein the method comprises receiving patient glucose level data indicative of glucose level information of the patient (in particular, also information with regard to planned meals and/or an insulin sensitivity level of the patient can be received), determining the insulin dosage in an interactive time-dependent manner based on applying the received patient glucose level data to at least only predefined insulin dosage determining criterion (in particular, the insulin dosage may correspond to a calculated daily dose), and outputting a result of the determining as a proposal for the insulin dosage indicative of doses and assigned times of the day according to which insulin is to be administered to the diabetes patient.

According to still another exemplary embodiment of the invention, a program element (for instance a software routine, in source code or in executable code) is provided, which, when being executed by a processor (such as a microprocessor or a CPU), is adapted to control or carry out a method having the above mentioned features.

According to yet another exemplary embodiment of the invention, a computer-readable medium (for instance a CD, a DVD, a USB stick, a floppy disk or a hard disk) is provided, in which a computer program is stored which, when being executed by a processor (such as a microprocessor or a CPU), is adapted to control or carry out a method having the above mentioned features.

Data processing which may be performed according to embodiments of the invention can be realized by a computer program, that is by software, or by using one or more special electronic optimization circuits, that is in hardware, or in hybrid form, that is by means of software components and hardware components.

According to an exemplary embodiment, a reliable architecture of treating a diabetes patient with insulin is provided, which is based on objective parameters, i.e. measured patient glucose levels and predefined insulin plan determining criteria, for supporting a user (such as a physician or a nurse in a hospital) in treating a diabetes patient medically correctly. Consequently, a failure-free planning of times and doses of administering or non-administering insulin to the patient becomes possible. Therefore, even in a hectic environment (such as a hospital) and without the need to necessarily consult a physician or focus specifically on the diabetes (for instance when the patient is in hospital due to another illness or injury, wherein the diabetes is considered only in addition), it is possible to ensure that a diabetes patient is reliably provided with the sufficient amount of insulin without the risk of overdosing and underdosing. Since basically no specific human medical skill is required for carrying out such an insulin dosage proposal by operating the device, the device can also be used by a person at home. In a hospital or any other medical facility, the system may be operated by a physician, but also by a non-physician, such as a nurse. In an embodiment, the main users are nurses, and only for therapy initialization and daily therapy adjustment a physician is consulted. Glucose measurement and individual insulin administration may be handled independently by a nurse. In an embodiment, the system may be equipped with a configurable user management feature which allows to adapt roles in accordance with present circumstances. Functionalities can be coupled to user roles in a configurable manner. Moreover, not only a single dose of necessary insulin based on a single glucose measurement is performed, but the proposed insulin dosage is based on a plurality of glucose level measurements performed at different times of the day, and the proposed insulin dosage is capable of distributing multiple doses over a period such as a day. Such a planned delivery of individual insulin doses at different times of the day in different amounts also allows for an adaptation of the insulin delivery plan to meet also other boundary conditions, such as other duties or appointments of the diabetes patient (for instance other treatments or diagnostic events in a hospital). For instance, it is possible that the device and method automatically adjust or adapt an insulin dosage plan in the event that, contrary to the proposed dosage, no insulin has been administered to the patient (for instance since the patient was absent at a proposed insulin administer time or time interval, for example due to a medical checkup or a required medical care), and the device and method will consider these boundary conditions and propose another insulin dosage meeting these boundary conditions.

### Overview of Exemplary Embodiments

The described electronic assembly is based on the idea that an electronic package comprising an electronic component, in particular a semiconductor electronic component, can be realized in an efficient manner by using a known printed circuit board (PCB) respectively PCB technology. When using at least one PCB all processing techniques which are already available for PCBs and for all the materials being used for PCB technology can be employed for realizing the described electronic assembly.

In the following, further exemplary embodiments of the device, the arrangement, the method, the program element, and the computer-readable medium will be explained.

In an embodiment, the output interface is further configured to display a workflow in accordance to which certain doses of insulin have to be administered to the patient, and is configured to indicate other activities (in particular the next glucose measurements to be performed and other events) related to a workflow in a medical facility. The insulin management system is therefore capable to integrate a workflow management in institutional care. In this context, the “patient” may denote the person to whom the insulin is administered. The “user” may be a person prescribing the insulin and/or adapting an insulin dosage (such as a physician), and/or may be a person measuring glucose level and/or administering insulin (such as a nurse), and/or may be the patient itself (for instance in an application at home).

In an embodiment, the device is programmed to monitor insulin supply and glucose levels of more than one assigned diabetes patient of the medical facility. As particularly advantageous in the environment of a medical facility in which the medical facility's workflow is considered for the insulin management, management of the insulin delivery for many patients at the same time by the system combines individual insulin delivery requirements of each individual patient with boundary conditions within the medical facility requiring to consider the needs of multiple patients at the same time.

In an embodiment, one of the at least only predefined insulin dosage determining criterion considers historic glucose level data of the patient (i.e. glucose level measurements performed with the patient in the past), in particular glucose level data of the patient of the preceding day, more particularly at least one of the group consisting of glucose level data of the patient in the morning of the present day and in the evening of the preceding day and glucose level data of the patient averaged over the preceding day (for instance averaged over three or four measurements of the preceding day). In such an embodiment, the prediction of the necessary insulin doses in the future is based on the knowledge of the time dependence of the glucose level of the patient in the past. Therefore, patient-specific physiological particularities may be considered when proposing the insulin dosage for the future. It has turned out to be particularly reliable to use the measured blood glucose levels of the patient under consideration from the present day in the morning and from the preceding day in the evening, and additionally the average blood glucose level over more than these two measurements, for instance averaged over four measurements (which may be performed in the morning, at midday, in the evening and in the night) of this patient.

In an embodiment, one of the at least only predefined insulin dosage determining criterion considers a historic insulin dose administered to the patient (i.e. insulin doses supplied to the patient in the past), in particular an (for instance overall) insulin dose administered to the patient during the preceding day. It has turned out to be an important criterion for proposing the insulin dosage for the next day which amounts of insulin have been administered to the patient in the past. Therefore, particularly in combination with the consideration of the historic glucose levels of the patient, the physiological response of the patient to the administration of insulin can be taken into account.

In an embodiment, one of the at least only predefined insulin dosage determining criterion considers a present amount of insulin in the body of the patient, in particular based on previously administered insulin doses and an extrapolation based on the assumption of a linear decrease of the insulin in the body of the patient. Therefore, the insulin on board, i.e. the insulin which is presently still within the body of the patient, can be also considered for proposing future insulin doses for this patient when assuming a certain decrease profile (for instance a linear decrease) of the insulin level after administration.

The described criteria are all easily applicable on the basis of either standard measurements or a documentation of the insulin delivery of the patient in the past. Hence, these and other criteria, alone or in combination, can therefore be easily implemented into a machine-based proposal system for an objective basis for a future insulin delivery strategy in terms of times and doses.

In an embodiment, the processor is configured for determining the doses separately for a long-term active insulin, in particular basal insulin, and for a short-term active insulin, in particular bolus insulin. The objective proposal of insulin delivery rates to be administered to the patient can also consider a combination of two (or more) kinds of insulin to be administered to the patient. A first kind of insulin may be insulin which is effective for a relatively long term, such as insulin being effective with an effective time constant of for instance 24 hours (so-called basal insulin). A second type of insulin which has a shorter effective time constant of for example 4 hours (so-called bolus insulin) may be added. Since these two types of insulin are metabolised by the human body with different time characteristic, basal insulin as a basic component and bolus insulin as a specific, patient- and situation-dependent addition may be considered for establishing the insulin dosage.

In an embodiment, the processor is configured for determining the dose for the long-term active insulin and the dose for the short-term active insulin with a constant predefined ratio, in particular a ratio 1:1 or substantially 1:1 (for instance a deviation from 1:1 can result of a rounding to integer doses of insulin or due to the need to add corrective bolus insulin to the patient in the event of inappropriate measured glucose levels). However, other ratios are possible as well.

In an embodiment, the processor is configured for determining insulin delivery doses concerning the short-term active insulin to be administered to the patient in at least three partial doses per day to be delivered to the patient at different times of the day (preferably prior to each meal). For example, it is possible to administer insulin doses of the short-term active insulin in the morning, at midday and in the evening (preferably always shortly before a corresponding meal), and optionally a fourth dose during the night. Additional insulin doses may be calculated and administered whenever deemed necessary by the users.

Insulin administration and dose calculation can be supported in any desired number per day (for instance there can be less than four times of insulin administration and dose calculation per day). It is possible to consider only three meals (morning, midday, evening) for the calculation. In case that a certain meal has already been considered for the calculation at a certain time of the day, it is possible that only an increased corrective dose is proposed, wherein possible insulin on board may be subtracted.

In an embodiment, the processor is configured for determining the at least three partial doses under consideration of the boundary condition that the doses of the short-term active insulin are unequal, i.e. are not administered in equal doses to the patient. Thus, a more precise adaptation to the physiology of the patient is possible. In other words, selecting different amounts of individual bolus doses may be used as a design parameter for further improving the treatment of the patient.

In a preferred embodiment, the processor is configured for determining the at least three partial doses under consideration of the boundary condition that the largest of the doses of the short-term active insulin is administered to the patient in the morning. In particular, the morning dose (i.e. administered for breakfast) shall then be larger than the midday dose (i.e. administered for lunch), provided that the basal insulin is administered at midday. It has been surprisingly found that providing the largest dose of short-term insulin in the morning (rather than at midday where in many cases the richest meal is ingested) results in a flat glucose curve in the patient and provides the patient with a comfortable feeling. More specifically, administering the largest short-term insulin dose in the morning, a significantly smaller amount at midday and an increased (i.e. intermediate) dose in the evening has turned out to provide medically advantageous results.

In a preferred embodiment, the processor is configured for determining the at least three partial doses under consideration of the boundary condition that a percentage of the short-term active insulin administered in the morning is in a range between about 40% and about 50% (in particular between 42% and 48%) of the entire short-term active insulin, a percentage of the short-term active insulin administered at midday is in a range between about 20% and about 30% (in particular between 22% and 28%) of the entire short-term active insulin, and a percentage of the short-term active insulin administered in the evening is in a range between about 25% and about 35% (in particular between 27% and 33%) of the entire short-term active insulin. Of course, all doses of the entire short-term active insulin administered during a day should sum up to 100%. In particular, the morning dose of short-term insulin can be advantageously in an order of magnitude of 45% of the entire day doses, can be at midday in the order of magnitude of 25% and can be around 30% in the evening. The described unequal distribution of the bolus insulin over the various daily doses seems to properly meet the medical requirements of the human physiology.

In an embodiment, the processor is configured for determining insulin delivery doses for the short-term active insulin so that each dose comprises a meal-depending base contribution and a patient-depending corrective contribution. Hence, apart from distinguishing between short-term active insulin and long-term active insulin, the short-term active insulin can further be separated into two components. A meal-depending base contribution can be dimensioned and administered at a corresponding time and in accordance with a respective meal of the patient (which can for instance be taken in the morning, at midday and in the evening). A corrective patient-depending contribution may be added additionally and may consider particularities of the physiology of the respective patient and which may also consider a presently measured glucose level. Therefore, a still simple but already sufficiently precise and individual composition of the short-term active insulin is possible. In particular, the patient-depending corrective contribution may be blood glucose dependent, i.e. may depend on a measured glucose value of the patient, and/or may be patient-type depending, for instance may depend on as to whether the patient is to be classified as insulin sensitive, normal or resistant.

In a highly preferred embodiment, the processor is configured for determining the at least three partial doses of the meal-depending base contribution under consideration of the boundary condition that a percentage of the meal-depending base contribution administered in the morning is in a range between 40% and 50% (in particular between 42% and 48%), a percentage of the meal-depending base contribution administered at midday is in a range between 20% and 30% (in particular between 22% and 28%), and a percentage of the meal-depending base contribution administered in the evening is in a range between 25% and 35% (in particular between 27% and 33%). The described unequal distribution of the meal-depending base contribution of the bolus insulin over the various daily doses results in an advantageously flat characteristic of the patient's insulin/glucose behaviour, while still allowing to flexibly adjust deviations of the measured glucose levels from desired values by adding corrective amounts of patient-depending corrective contribution of bolus insulin.

In an embodiment, the meal-depending base contribution has always a positive value. In an embodiment, it may have an absolute value which is larger than an absolute value of the patient-depending corrective contribution. It has turned out that the meal-dependent contribution is in many cases larger than the patient-dependent contribution, since the latter usually has a smaller effect as compared to the usually larger impact of the meals.

In an embodiment, the patient-depending corrective contribution selectively has a positive value or a negative value. By allowing the patient-depending corrective contribution to assume any desired positive or negative value, it may even be considered if the physiology of the patient requires to reduce the meal-dependent contribution by a subtraction of administered insulin, i.e. by adding a negative corrective value. This can be particularly appropriate for patients being very sensitive in terms of changes of the insulin level on board with respect to the corresponding glucose levels. For certain patients presently showing a very low glucose level, a reduction of insulin is particularly appropriate when the measured glucose level is relatively low, for instance below a certain threshold value. Different sub-ranges of glucose levels of such kind of patients may be distinguished in which the absolute value of the negative contribution is different. Such a correction involves only a very small additional computational burden on the insulin dosage determination but precisely reflects present medical particularities of such a patient. For less sensitive patients (a certain classification can be made in form of a look-up table or the like, in which a sliding scale may be implemented), the concept of negative insulin contributions may be optionally omitted.

In an embodiment, the patient-depending corrective contribution (also denoted as correction insulin) may be determined on a sliding scale. For instance, a table with correction insulin amount per blood glucose range may be provided, for example uniformly ascending. For example, insulin sensitive patients get −2 insulin units for taking into account this sensitivity, whereas insulin resistant patients get +2 insulin units for taking into account this resistance.

In a further embodiment, the patient-depending corrective contribution may be determined by multiplying a respective insulin base dose by a factor (which may be larger or smaller than one and which may, for example, depend on the patient's insulin sensitivity or insulin resistance). For determining the factor, it is possible to use one or a combination of the insulin sensitivity or resistance, the HbA1c value (or another marker or indicator for long term glucose level), the total daily dose, and/or the treatment day.

Glycated hemoglobin (HbA1c) is a form of hemoglobin that allows to identify the average plasma glucose concentration over prolonged periods of time. Any other long-term marker (or indicator) of average glucose concentration can be used instead as well.

In an embodiment, the processor is configured for determining an insulin delivery dose concerning the long-term active insulin to be administered to the patient once a day. In contrast to the supply of the short-term active insulin several times a day, it may be sufficient—and then simplifies the insulin dosage—to provide the long-term active insulin only once a day, for example directly before lunch. This further simplifies the integration of the insulin dosage into a workflow of a medical facility such as a hospital or into the all-day life needs of a user operating the device on her or his own.

In an embodiment, the processor is configured for determining an initial insulin delivery dose, in particular an initial daily insulin delivery dose, to be administered to the patient at the first time of proposing an insulin dosage for this specific patient based on one or more initial dose determining criteria, in particular based on age, weight and creatinine level (i.e. a value indicative of the kidney function of the patient) of the patient. Thus, a computationally very simple basis is provided for calculating the initial or first insulin dose at the first day a diabetes patient is treated with the computer-implemented device or the computer-implemented method. Parameters such as age, weight and creatinine level can be obtained by the device via data communication with a hospital patient information database (which may form part of the control entity or may be accessible by the control entity) in which these data items are usually stored for the patients of a medical facility. Again, a medically reasonable first day estimation is possible based on these parameters.

The initial daily insulin delivery dose (also denoted as initial total daily dose) is important, because the more reliable it is, the better is the glucose control from the very beginning onwards. A simple approach for determining the initial daily insulin delivery dose according to a first exemplary embodiment of the invention is to assume a predetermined value of insulin unit per body weight of the patient (for instance 0.5 IU/kg body weight). For patients with a certain medical profile or characteristic (for instance a serum creatinine level above a predetermined threshold value, for instance above 2.0 mg/dl, and/or with an age above a threshold value, for instance at least 70 years), a reduced predetermined value of insulin units per body weight of the patient may be defined (for instance 0.3 IU/kg body weight).

In a more refined embodiment, one or more additional parameters may be considered for determining the predetermined value of insulin units per body weight of the patient. These parameters may be the body mass index, a previous insulin treatment, an HbA1c value, the presence of an acute hospitalization and/or a severe illness, a target range, etc.

In an embodiment, the processor is configured for determining a subsequent insulin delivery dose (for instance a dose for the second day of operating the device for the patient) to be administered to the patient after administering the initial insulin delivery dose based on the initial insulin delivery dose and based on glucose level data of the patient. Therefore, by an iterative procedure, the dose from the first day may be used as a starting point for calculating next day doses, wherein the physiological reaction on the insulin supply, as reflected by the actual glucose measurements of the patient, can be considered for adjusting the doses for the next day.

In an embodiment, the processor is configured for determining a next day insulin delivery dose to be administered to the patient after having administered a preceding day insulin delivery dose based on the preceding day insulin delivery dose, based on glucose level data of the patient at the preceding day and at the present day, and based on an estimated amount of insulin being presently within the body of the patient. It has turned out as a very reliable and medically reasonable way of calculating a next day insulin dose based on the insulin delivery proposal plan of the previous day and by additionally considering, in an iterative way, the glucose levels measured yesterday evening and in the morning of the present/current day. The consideration of these parameters has turned out to be sufficiently precise to predict which amount of insulin is reasonable for the current day, i.e. for the next 24 hours.

In an embodiment, the processor is configured for determining a future insulin delivery dose, in particular of bolus insulin, to be administered to the patient under consideration of an estimated amount of insulin being presently within the body of the patient. Hence, the dosage recommendation for the short-term active insulin may be corrected based on the insulin on board value, i.e. the value of previously administered but still active insulin.

In an embodiment, the processor is configured for correcting a late-administered insulin delivery dose administered to the patient with time delay, in particular of basal insulin, under consideration of the time delay. Thus, the dosage recommendation for the long-term active insulin, which is delivered too late as compared to the intended timeframe (or compared to an insulin delivery plan), can be corrected (i.e. reduced) depending on the time of the day (at which the insulin is in fact administered). For instance in a scenario in which basal insulin is administered too late, the insulin can be reduced in accordance with this delay time interval. For example, if insulin is administered four hours too late, the calculated dose of basal insulin (for 24 hours) can be reduced by 4/24=16%.

In an embodiment, a dosage of long-term active insulin may be reduced on the first day. In one alternative, only a warning is displayed to the physician, but the reduced basal insulin dose cannot be ordered explicitly. In another embodiment, the basal insulin dose may be reduced for newly initialized patients dependent on the time of the day and recently administered insulin and/or other medication. In this context, it should be considered that at the first day at the medical facility, the patient has probably already received insulin (which may also comprise long-term active insulin). If this is the case, the insulin dosage of basal insulin should be reduced with the next administration. Since in many cases a physician is not present when the next dose is administered to the patient by a nurse, the described phenomenon can already be considered with the initial prescription under control of the physician.

In an embodiment, the input interface is configured for receiving medical facility schedule data indicative of a schedule of treating patients in a medical facility, wherein the processor is configured for determining the insulin dosage in accordance with the schedule excluding scheduling conflicts concerning treating the patients in the medical facility and administering insulin to the patient. For instance, general fixed times (such as times of breakfast, lunch and dinner) of the medical facility may be input to the device. Hence, such fixed times of the medical facility may be considered for determining appropriate time intervals of measuring glucose level or administering insulin. The device and method may define time intervals for glucose measurements (for instance that the measurement in the morning shall be performed between 6 AM and 9 AM). By taking this measure, it is possible to integrate into the insulin dosage proposal system boundary conditions of a facility within which the insulin administration is performed. In an embodiment, the times of administering insulin are not patient dependent, but system dependent (for instance are configured once and fixed afterwards). For example, in a hospital, a patient suffering from diabetes and a further disease primarily requires a treatment of this further disease. For example, an X-ray measurement has to be performed for this patient at a certain point of time. Or, a consultation with a physician has to be performed at a certain time of the day. Since the device and method can dynamically react on a non-compliance with a proposed dosage plan (for instance the event that the proposed insulin has not been administered to the patient in due time) with an adapted proposal for the administration of the insulin to the patient, the use of the device and the method is compatible with the requirements in the hospital. For instance, a missed administration of the insulin can be caught up later, i.e. after the patient has returned from the X-ray measurement or consultation.

In an embodiment, the device and method make a dosing proposal in a time-dependent manner (interactively, in real time). More specifically, the processor may be configured for determining the insulin dosage in the interactive time-dependent manner directly responsive to a user input via the user interface and/or to an insulin administration and/or to one or more glucose measurements and/or to a present time of the day (the latter criterion may be correlated with the insulin on board, a time distance to the various meals, etc.). In this context, the term “interactively” may particularly denote directly reacting to a user input and/or to an insulin administration and/or to a glucose measurement and/or to the time of the day (which may have an impact on the insulin on board, and time distances to the various meals). This may include, if desired or required, consideration of insulin on board and/or other parameters.

In an embodiment, the device is configured as a portable device, in particular as a tablet (in particular a tablet having a touchscreen, the latter serving as both input interface as well as output interface). In such a tablet, the touch screen may allow to receive user commands or data input by the user, for instance a manually input glucose level data resulting from a blood glucose measurement. The display of the tablet allows to display the insulin dosage proposed by the computer-implemented device. The processor of conventional tablets is sufficient to carry out the relatively simple computational tasks of the insulin dosage proposal system. However, as an alternative to a tablet PC, it is also possible to realize the device to form part of a mobile phone, a watch-like device, a headset, etc.

By integrating the device(s) into a wireless communication system, it is possible to have one central computer with a large amount of processor capability and a large amount of storage capability and to use this control entity for centrally controlling multiple devices simultaneously. Multiple devices of the above-described type can all be configured relatively simple because they may use (at least in addition) the central resources in terms of processor capacity and/or storage capacity of the control entity. Therefore, the clients in form of the devices can be used at different wards/departments of a hospital or the like, all being served by a common control entity as server.

In an embodiment, the devices and the control entity are configured for a wireless communication, in particular for communicating via WLAN. Therefore, data required for calculating an insulin dosage by the devices, can be wirelessly transmitted from the control entity to the devices making use of a hospital data information system being centrally available. Therefore, the entire data storage capability of the arrangement may be relatively small, since only very specific patient data can be downloaded from the control entity towards the devices.

In an embodiment, the control entity comprises a patient database storing medical data of the plurality of patients, wherein the plurality of devices are configured for determining the insulin dosage under consideration of the medical data of the respective patient. For example, this may include age, weight and creatinine level of the patients. Based on these values, the initial daily insulin dose after using the device for the first time for a specific patient may be calculated.

In an embodiment, the control entity comprises a program database storing program code for determining the insulin dosage, wherein the plurality of devices are configured for displaying the insulin dosage determined by executing the program code. Therefore, also the program code needed for executing the computer-implemented method and for operating the computer-implemented device may be downloaded (for instance for updating) by the devices from the control entity, more precisely from its database. Therefore, the software needed for operating the devices may be at least partially provided from the control entity. However, it is possible in one embodiment to store a part of the required software on the devices, for instance in form of a downloadable app. It is also possible in another embodiment to store the entire required software on the devices.

It is also possible that the control entity (as server) serves for performing at least part of the calculations (in particular the entire calculations) in terms of proposing insulin dosages, and the devices (as clients) receive the results transmitted from the control entity for display. When the control entity performs the calculations, it is sufficient that only one central intelligent entity is provided which does the main part of the processing tasks. No synchronization of redundant data between many entities is necessary in such a scenario. However, it is alternatively also possible that the calculations are all done locally by the devices, which renders the arrangements less prone to failure for instance in a scenario in which a WLAN connection between the control entity and the devices fails.

Each of the program database and the patient database may be embodied as a mass storage apparatus (such as a hard disk) storing the respective data. In one embodiment, both databases are embodied by one common mass storage apparatus, whereas in other embodiments the two databases are arranged on two separate mass storage devices.

In an embodiment, the method is applied for diabetes patients of a hospital. Therefore, many patients in a hospital can be reliably treated with regard to their diabetes disease in addition to another disease for which reason the patients are actually in the hospital. However, it is also possible to implement the computer-implemented device and method in another medical facility environment such as a retirement home.

In an embodiment, the method is applied for a diabetes patient at home. Since no physicians are required to operate the device and computer-implemented method, it is also possible that a patient at home uses the device and the computer-implemented method for determining an insulin dosage for himself. The only required input are patient data such as age, weight, etc. and blood glucose measurement values which can be provided by a patient itself. For such an application at home, it is possible that the insulin dosage is sent, via a communication network (such as the Internet), also to a physician for control purposes. Even in a medical facility such as a hospital, it is possible that the calculated insulin dosage proposals of the devices are stored centrally in the control entity for documentation purposes or the like.

In an embodiment, the processor is configured for triggering a reminder (for instance visual reminders, audio reminders and/or haptic reminders) to be output by the output interface upon identifying a lacking execution of an action required in the framework of the insulin dosage. Such reminders may be sent for executing glucose level measurements and inputting their results to the device, confirming the supply of insulin doses in accordance with the proposed (and accepted) insulin dosage, completion of a corresponding medical treatment of the patient in the medical facility in accordance with the schedule, etc.

In an embodiment, the processor is configured for deactivating the insulin dosage (and the process of continuously proposing and updating the plan) upon identifying a continued lack of execution of the reminded action in spite of the reminder. If the device identifies non-compliance with the proposed and accepted insulin dosage (for instance although a reminder has been issued and an additional period has expired without completing the omitted act), the device can no longer guarantee safe treatment of the patient and will therefore terminate the process. Since in this case no reliable dose proposal may be made, the system deactivates to increase patient's safety. For example, only a user having a certain authorization (for instance a physician) may reactivate the device after such an automatic deactivation.

In an embodiment, the processor is configured for deactivating the insulin dosage upon identifying lack of data required for a reliable dosage recommendation. Thus, the dosage proposal system may be terminated when required data are missing, such as glucose measurement on the patient in due time, and/or insulin administration to the patient in due time. Hence, if a user of the device and method does not comply with the requirements of the device or method by not performing glucose measurements in reasonable time intervals and/or by not performing the (daily) therapy adjustment within the intended time interval, the device and method may transit into an inactive mode due to lack of reliability in view of the lack of compliance of the user. Additionally or alternatively, the device and method may transit into an inactive mode (i.e. the decision support may be deactivated) in a scenario in which a value which has already been used for a calculation of the insulin dosage has been later changed or corrected by a user.

In an embodiment, the processor is configured for, before executing a received user command, verifying a user authorization concerning the user command by determining whether the user command complies with user authorizations defined in a corresponding user profile. It is hence possible that the device comprises a database or is capable of accessing a database of the control entity which includes an authorization profile for different users of the device, for instance distinguishing between physicians and nurses in the hospital. For instance, the database may be located at a central position in the hospital, for instance may form part of the hospital database. Depending on a role of the user (for instance role “physician” or role “nurse”), the authorization to access data (for instance patient data in the database), the authorization to input data (such as measured glucose levels for patient) and/or the authorization to make decisions may be granted or denied in accordance with the specification of the assigned user profile (which may for instance be more extensive for the role “physician” than for the role “nurse”).

The method may be configured as a computer-implemented method, to be executed by a processor, without the need to involve a medical expert such as a physician at all (if allowed by a specific jurisdiction). For jurisdictions in which it is legally required, it is possible to configure the system in such a manner so as to involve the physician to the extent required by the corresponding law. For example, approval of a readily proposed dosage scheme can be performed by a physician. The execution of the therapy can be performed by nurses or a patient at home. In an embodiment, a proposed dosage needs only final confirmation or approval by a user such as a physician or a nurse, before being applied to a patient.

In an embodiment, a decision support system for use in a hospital or in another environment is provided which may serve as a basis for a user to estimate an amount of insulin to be delivered to a patient. Therefore, an active system is provided for calculating a time dependence of an insulin delivery for a patient under consideration of the operation schedule of a medical facility. The general workflow in the medical facility as well as the workflow for executing the computer-implemented method and for operating the computer-implemented device may be also centrally stored in the control entity, more precisely in a database thereof. Configuration data of the workflow in the medical facility may be stored in the database, such as times or time intervals at which meals are provided to the patients, times or time intervals at which glucose measurements shall be carried out, and/or times or time intervals at which insulin shall be administered. Individual activities, such as glucose measurement and/or administering insulin, can be varied with a certain flexibility.

In an embodiment, the workflow of the device may be stored in a user front end device, for instance a tablet. Configuration data (for instance times of the day) may be stored in a database.

By connecting the (in particular mobile) devices with a hospital and laboratory information system, manual and multiple inputs may be avoided, rendering the system failure robust and easy to use.

One concrete exemplary algorithm according to which the device or the computer-implemented method determines an insulin dosage and individual doses to be administered at different times during a day may operate as follows: An initial overall insulin amount for 24 h can be computed based on patient weight, patient age and renal function. For every subsequent day, a new overall insulin amount for the next 24 h can be calculated based on the overall insulin amount of the preceding day, the breakfast glucose level of the current day and the dinner glucose level of the day before and an average value of all glucose levels of the day before. Also, the entire insulin value present in the body of the patient may be considered for this calculation and may be estimated based on a reasonable model of reduction of insulin (for instance based on the assumption that administered insulin reduces in the body of the patient with a linear decrease). Then, the calculated overall insulin amount for a day can be divided into a 50% basal insulin dose and a 50% bolus insulin dose to cover the intended three meals (breakfast, lunch, dinner). If the calculated basal or bolus dose results in a fraction, it may be rounded to the next lowest integer. At or around midday, the basal insulin dose can be administered as the long-acting insulin analogue. The bolus insulin dose can be further divided into three unequal doses, the morning dose being larger (e.g. 45%) than the midday dose (e.g. 25%) and the evening dose (e.g. 30%) being in between the morning dose and the midday dose. If these bolus doses are non-integer values, the remaining insulin units may be added to breakfast and midday bolus, because particularly appropriate results may be achieved with the described unequal distribution with the largest supply of bolus insulin in the morning. In case measured pre-meal glucose values are out of target range, the suggested bolus dose may be adjusted further by using insulin sensitivity and the current glucose level (corrective bolus dose).

In an embodiment, the processor is configured for determining a daily insulin dosage for a respective full day. The output interface may be correspondingly configured for outputting the proposal for the insulin dosage indicative of multiple doses for the full day which sum up to the determined respective daily insulin dosage. Therefore, the system may support the adaptation of the daily insulin doses for a user (which can be calculated for each new day in advance), and all dosage recommendation may be based on this daily based administration scheme. Parameters may be selected for the adaptation of the daily dose.

When determining a daily insulin dosage (also denoted as total daily dose), it is possible that the daily insulin dosage is increased or decreased in predefined steps (of for instance of ±10%). If the blood glucose level in the morning and in the evening is above a predefined value (of for instance 180 mg/dl), the step may be increased (to for instance 20%).

However, it may happen for some patients that the daily insulin dosage is determined to be much too low (or much too high). In such an event, it may be beneficial to increase (or decrease) the daily insulin dosage more aggressively. Such a scenario in which the daily insulin dosage is to be increased (or decreased) more aggressively as compared to the above-described concept may be determined based on one or a combination of at least two of the following parameters: body mass index, HbA1c value, previous insulin dosage, diabetic duration, and/or acute illness.

In a preferred embodiment, the processor is further configured for determining a home insulin therapy regimen and an insulin dosage to be administered to a diabetes patient of the medical facility after discharging the patient from the medical facility (for example to the patient's home, to a retirement home, to a rehabilitation facility, etc.) and following the insulin dosage to be administered to the diabetes patient in the medical facility. In this context, the term “therapy regimen” may particularly denote a decision which kind of insulin (in particular pure long-term active insulin, pure short-term active insulin, or pre-mixed insulin with a certain ratio between long-term active insulin and short-term active insulin) is to be used for the therapy, whereas the “insulin dosage” defines which amount of insulin should be administered at a certain point of time in accordance with the selected therapy regimen. The interface between the medical facility (with its high performance medical resources) and the patient's home (where the patient is substantially on his own and is only temporarily supported by his doctor) is critical in terms of a consistent therapy concerning insulin dosage. This should be considered in view of the very different resources of medical knowledge available for a patient in a medical facility such as a hospital and at home, but also should be considered in view of the available freedom (which is high in the medical facility and lower at home) in configuring the administered insulin (for example, different doses of insulin may be administered to a patient in the medical facility, wherein pure long-term active insulin may be used for one dose and pure short-term active insulin may be used for another dose, with a freely selectable ratio of short-term active insulin and long-term active insulin to be administered to the patient over multiple doses). Therefore, a continuous transition of the insulin administration at the medical facility and the insulin administration at home is of importance for the patient's health. The device and method may hence consider such different levels of medical resources at the medical facility and at home and may therefore calculate a medically safe proposal for a medical doctor concerning a transition between the treatment at the medical facility and the following treatment at home.

More specifically, the processor may be configured for determining the home insulin dosage with a fixed ratio between a long-term active insulin, in particular basal insulin, and a short-term active insulin, in particular bolus insulin, whereas the insulin dosage at the medical facility is determined with a variable ratio between the long-term active insulin and the short-term active insulin. Depending on the selected home therapy regimen, the mentioned fixed ratio may be 100:0 (i.e. basal insulin only), 0:100 (i.e. bolus insulin only) or may assume any selectable value in between these two extremes (such as in case of pre-mixed insulin with a constant ratio between long-term active and short-term active insulin). Usually, the resources at a medical facility such as a hospital allow to administer to the patient variable percentages of long-term active insulin and short-term active insulin. However, this concept is often too complicated for a patient at home (even when supported by a medical doctor caring for the patient from time to time), so that it is usually more reliable from a medical point of view to provide the patient at home with a simplified therapy regimen (such as a fixed mixture of short-term active insulin and long-term active insulin, wherein the only free parameter at home being the entire insulin amount without a chance to adapt the ratio between long-term active and short-term active insulin; or basal insulin only; or only bolus insulin with the meals).

In an embodiment, the processor is configured for (and the method comprises) determining the proposal for the insulin dosage by:
receiving, for instance via the input interface, at least one parameter indicative of a (in particular personal and/or medical) status of the diabetes patient;
determining or defining (for instance based on clinical guidelines) a target value of a long-term marker (or indicator) of average glucose concentration of the diabetes patient (such as HbA1c);
determining a blood glucose target range based on the target value of the long-term marker of average glucose concentration;
receiving, for instance via the input interface, data indicative of a home therapy of the diabetes patient; and
determining a therapy (i.e. a therapy regimen and an insulin dosage) for the diabetes patient appropriate for achieving the target value and/or the target range (in particular, the therapy regimen at the medical facility may be different from another therapy regimen after discharging the patient from the medical facility).

Thus, it is possible according to an exemplary embodiment to recommend a blood glucose target range and a therapy regimen. In this context, it is possible to collect one or more parameters on the patient's status (such as age, life expectancy, diabetes duration for more than a predetermined threshold value such as 10 years, a cardiovascular disease, recurrent severe hypoglycaemic events or impaired perception of hyperglycaemia, patient competence and resources, and/or highly elevated serum creatinine). A long-term HbA1c goal may then be recommended (for instance based on medical guidelines). Based on this HbA1c goal, it is then possible to compute a blood glucose target range. For instance, a corresponding service may be provided as a wizard on the device. Moreover, it is possible to collect data on the home therapy of the patient. On the basis of this data, it is possible to recommend a therapy (or therapy regimen) suitable for the patient to achieve the treatment goal defined above.

This recommendation may consider one or more of the parameters collected above and the treatment goal. The recommendation may comprise a suitable therapy for the patient at home. If required (for example due to the severity of illness or practicability in the hospital), a different therapy may be used during the hospital stay.

The recommended therapy may comprise or consist of a therapy regimen and a blood glucose target range. The blood glucose target range may affect correction insulin dose recommendations by the algorithm. It is also possible that the target range in the basal-bolus algorithm is fixed.

It is possible that the current therapy can remain unchanged if a blood glucose target is reached, no relevant contraindications are present and/or no significant hypoglycaemic events are present.

A discharge therapy regimen may be recommended correspondingly. The therapy success in the hospital or other medical facility may be considered for this recommendation.

In an embodiment, the system may be used to compute an initial daily dose of insulin (and in particular a basal insulin dose) for use with an insulin patch pump. More generally, exemplary embodiments of the invention may be used in terms of a subcutaneous insulin delivery as injection, as well as for an insulin infusion.

### BRIEF DESCRIPTION OF THE DRAWINGS

The aspects defined above and further aspects of the invention are apparent from the examples of embodiment to be described hereinafter and are explained with reference to these examples of embodiments.

FIG. 1 illustrates an arrangement for managing insulin delivery to a plurality of diabetes patients of a medical facility according to an exemplary embodiment of the invention.

FIG. 2 illustrates a scheme in accordance with a proposed insulin dosage according to which insulin is to be administered to a diabetes patient according to an exemplary embodiment.

FIG. 3 illustrates a block diagram showing a flow chart according to a method of proposing an insulin dosage to be administered to a diabetes patient according to an exemplary embodiment.

FIG. 4, FIG. 5, FIG. 6, FIG. 7, FIG. 8 and FIG. 9 show different operation modes of a device for proposing an insulin dosage for administration of insulin to a diabetes patient according to an exemplary embodiment of the invention.

### DETAILED DESCRIPTION OF THE ILLUSTRATED EMBODIMENTS

The invention will be described in more detail hereinafter with reference to examples of embodiment but to which the invention is not limited.

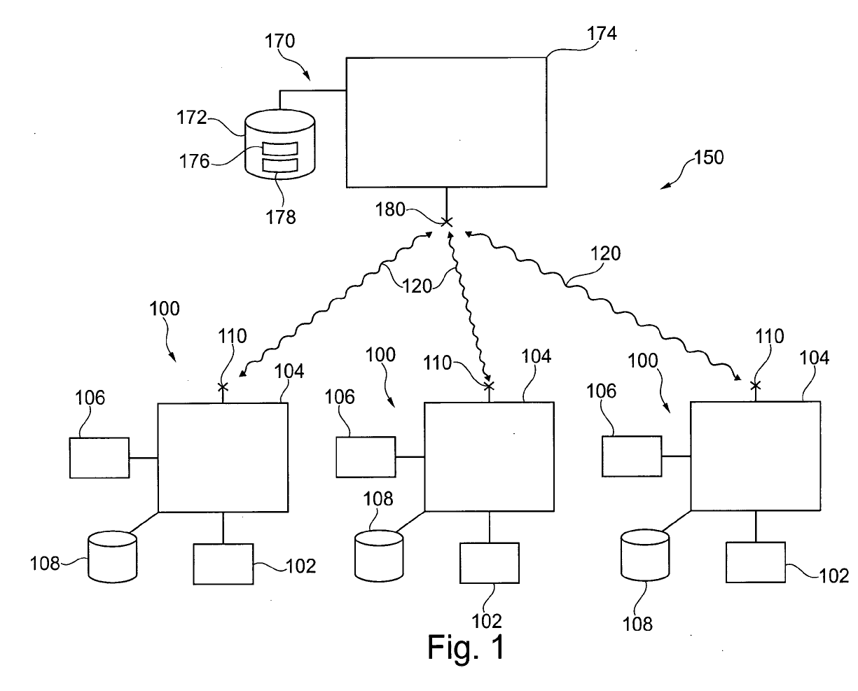

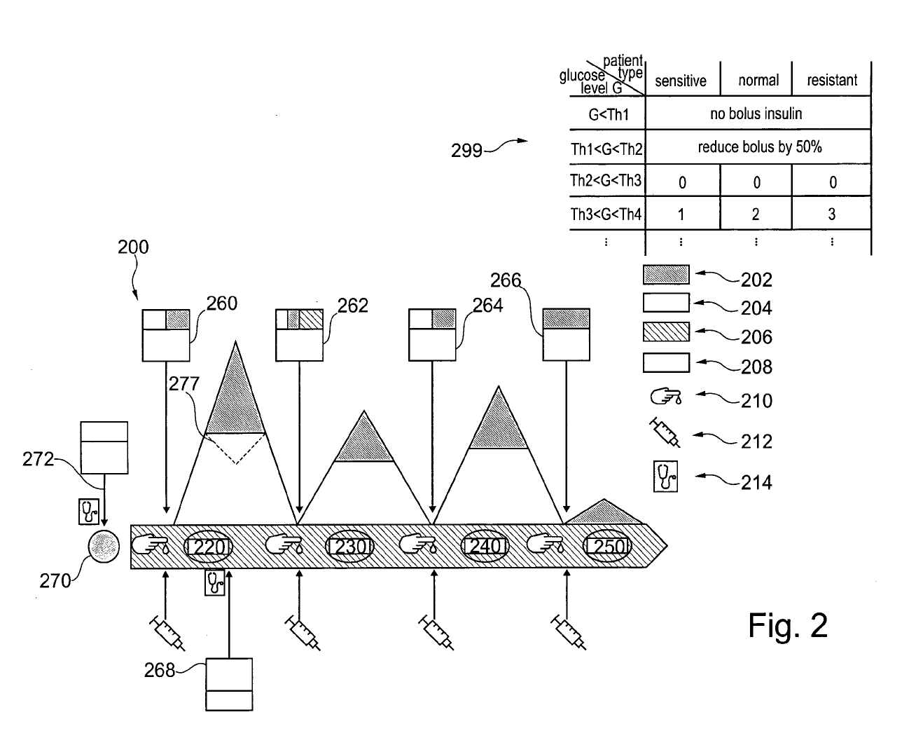

FIG. 1 illustrates an arrangement 150 for managing insulin delivery to a plurality of diabetes patients of a medical facility (such as a hospital) according to an exemplary embodiment of the invention. FIG. 2 illustrates a correspondingly proposed insulin dosage 200 according to which insulin is to be administered to a corresponding diabetes patient according to an exemplary embodiment.

The arrangement 150 shown in FIG. 1 is configured as a communication network which comprises a plurality of devices 100, which may be embodied as tablet PCs, each for proposing an insulin dosage 200 to be administered to an assigned one of diabetes patients of the medical facility. The devices 100 are configured for a wireless (unidirectional or preferably bidirectional) communication via WLAN with a control entity 170 for exchanging control data and patient data between the control entity 170 and the devices 100. For this wireless communication which is indicated schematically with reference numeral 120, each of the devices 100 comprises a corresponding communication interface 110, and the control entity 170 also comprises a correspondingly configured communication interface 180. A storage unit 108, such as a hard disk, may be provided for each of the devices 100 as well.

Each of the devices 100 comprises an input interface 102, for instance the touchscreen of the tablet PC, configured for receiving patient glucose level data 210 indicative of glucose level information of the patient. The glucose level data 210 may be input manually by a user of the device 100, via the touchscreen, and may indicate the result of a glucose measurement performed on the patient. A processor 104 of each of the devices 100 is configured for determining the insulin dosage 200 based on the received patient glucose level data 210 under consideration of one or more predefined insulin plan determining criteria (which may use the measured glucose levels and additional information, such as previously supplied amounts of insulin). An output interface 106 of each of the devices 100, which also may be embodied as the touchscreen of the tablet PC, is configured for outputting a result of the determining as a proposal for the insulin dosage 200 indicative of doses and assigned times of the day according to which insulin is to be administered to the diabetes patient. In other words, the output interface 106 displays for a user a workflow in accordance to which certain doses of insulin have to be administered to the user, and may also indicate the user other activities such as the next glucose measurements to be performed and other events related to a workflow in the medical facility.

The control entity 170 comprises a processor 174 for performing processing tasks in accordance with the control of the devices 100. The control entity 170 furthermore comprises a database 172 (which may be embodied as a hard disk) storing general medical data 176 of the plurality of patients, such as age, weight, gender and creatinine level. The plurality of devices 100 are configured for accessing the database 172 for determining the insulin dosage 200 under consideration of the general medical data 176 of the respective patient. Furthermore, the database 172 stores program code 178 for determining the insulin dosage 200. The plurality of devices 100 are configured for accessing the database 172 for determining the insulin dosage 200, for instance by executing the program code 178, which may for instance be downloaded by the devices 100. Alternatively, the program code 178 may be executed on the processor 174 of the control entity 170, and the results of the calculation may be transmitted to the respective device 100 to form at least part of the corresponding insulin dosage.

The input interface 102 of each of the devices 100 is configured for receiving medical facility schedule data 214 (which will be described below in more detail referring to FIG. 2) indicative of a schedule of treating the patient in the medical facility. The processor 104 is then configured for determining/adjusting the insulin dosage 200 in accordance with the schedule. This avoids collisions between medical appointments of the patient and times of the day at which activities (such as glucose measurement or insulin administration) are required in terms of the monitoring of the diabetes disease of the patient.

Now turning to FIG. 2, the processor 104 of each of the devices 100 is configured for determining an initial overall insulin amount for 24 h for a respective patient based on patient weight, patient age and renal function. For every subsequent day, a new overall insulin amount for the next 24 h can be calculated based on the overall insulin amount of the preceding day, the breakfast glucose level of the day before and the dinner glucose level of the day before and an average value of all glucose levels of the day before. Also, the entire insulin value present in the body of the patient may be considered for this calculation and may be estimated based on a reasonable model of digesting insulin (for instance based on the assumption that administered insulin reduces in the body of the patient with a linear decrease).

The processor 104 of each of the devices 100 is configured for determining insulin doses separately for a long-term active insulin 206 (“basal insulin”) and for a short-term active insulin 202, 204 (“bolus insulin”). The calculated overall insulin amount for a respective day can be divided into a 50% basal insulin dose and a 50% bolus insulin dose.

The processor 104 is configured for determining insulin delivery doses concerning the short-term active insulin 202, 204 to be administered to the patient in four partial doses to be delivered to the patient at different times of the day, i.e. in the present scenario in the morning 220, at midday 230, in the evening 240 and in the night 250. Moreover, the processor 104 is configured for determining an insulin delivery dose concerning the long-term active insulin 206 to be administered to the patient once a day, i.e. at midday 230.

The processor 104 is furthermore configured for determining insulin delivery doses for the short-term active insulin 202, 204 so that each dose may comprise a meal-depending base contribution 204 and a patient-depending corrective contribution 202. Since the patient does not take a meal during the night 250, the dose of bolus insulin administered at night 250 only comprises a patient-depending corrective contribution 202, and the meal-depending base contribution 204 is here zero.

Advantageously, the processor 104 can be configured for determining the partial doses under consideration of the boundary condition that the largest of the doses of the meal-depending base contribution 204 is administered to the patient in the morning 220. Preferably, a percentage of the meal-depending base contribution 204 administered in the morning 220 is in a range between 40% and 50%, a percentage of the meal-depending base contribution 204 administered at midday 230 is in a range between 20% and 30%, and a percentage of the meal-depending base contribution 204 administered in the evening 240 is in a range between 25% and 35%. The meal-depending base contribution 204 has always a positive value (unless there is no meal at all, i.e. at night 250) and may be fixed in accordance with a specific meal which the patient takes (i.e. separately for breakfast, lunch and dinner).

The patient-depending corrective contribution 202, which can assume values derivable from a lookup table 299, may have a positive value, may have a zero value or may have a negative value (as indicated by reference numeral 277). The actual value of the patient-depending corrective contribution 202 may depend on a measured glucose level G of the patient and on the patient's particularities, i.e. whether the patient is sensitive, normal or resistant. For instance, it can have a negative value in case the glucose level G of the patient, as measured, is below a first threshold value Th1. It can have another negative value with a smaller absolute value (e.g. 50%) in case the glucose level G of the patient, as measured, is above the first threshold value Th1 but below a second threshold value Th2. It can have a zero value in case the glucose level G of the patient, as measured, is above the second threshold value Th2 but below a third threshold value Th3. It can have a positive value in case the glucose level G of the patient, as measured, is above the third threshold value Th3 (wherein further sub ranges with corresponding values for the patient-depending corrective contribution 202 may be defined). For each of the defined sub ranges of measured glucose levels the absolute value of the patient depending corrective contribution 202 can be selected depending on whether the individual patient under treatment is sensitive (i.e. reacts strongly on physiological changes), has a normal behavior or is resistant (i.e. reacts in a week manner on physiological changes).

In FIG. 2, times of the day at which glucose measurements are performed with the patient, are indicated by reference numeral 210. Times of the day at which insulin is administered to the patient in accordance with proposed doses and assigned times of the day/in accordance with a proposed insulin dosage (compare reference numeral 260, 262, 264, 266, 268, 272) are indicated by reference numeral 212. Times of the day at which doctor's activities/medical activities occur concerning the patient, are indicated by reference numeral 214 (which may be denoted as medical facility schedule data). The admission of the patient to the medical facility/insulin dose proposal system is indicated schematically with reference numeral 270.

FIG. 2 hence shows a workflow for the treatment of patients with diabetes type 2 at a general ward. It incorporates workflow requirements of the clinical personnel as well as a decision support for insulin dosing. The decision support helps nurses and clinicians to find the proper dosage for their patients. The process starts with the admission of a patient at the general ward (compare reference numeral 270). Relevant patient and treatment parameters are automatically transferred from the hospital information system to the developed software-implemented into a dosage proposal system. Only patients who fulfil pre-defined medical parameters (e.g. diagnosis of diabetes type 2, creatinine level below a specific value, no gestational diabetes) will be enrolled for the glucose management and decision support system. During the enrolment process various initial parameters related to the medical status and the therapy of a patient can be specified by the clinician. The decision support may provide at this state of the workflow the clinician with an initial daily insulin dose based on age, weight and creatinine level of the patient. After therapy initialization four times per day blood glucose is measured before meal (compare reference numeral 210) and the decision support suggests the proper bolus units based on former blood glucose measurements. If the dosage is acceptable for the decision maker (such as a user of one of the devices 100) the insulin units are administered (compare reference numeral 212). The total daily insulin dosage is composed by the basic bolus insulin and the basal insulin. The basal insulin is admitted once a day at midday. Depending on the current blood glucose value supplement bolus insulin is calculated by the decision support. FIG. 2 shows the composing of the different insulin (basal, base bolus, supplement bolus) provided as dosage advice by the decision support.

Once per day, at the ward round, an adjustment of the therapy may be performed and a new daily insulin dose may be suggested by the decision support. The new dosage may be based on the old dosage and former blood glucose values (for instance morning and evening values of the previous day) of the patient. This daily insulin dosage sets the new basal and bolus insulin dose.

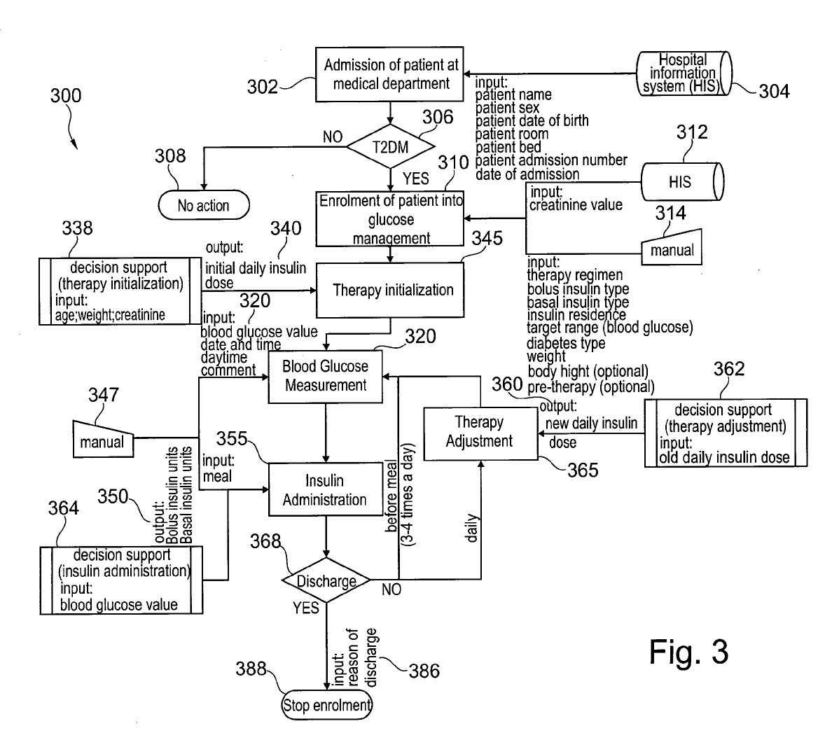

FIG. 3 illustrates a block diagram showing a flow chart according to a method 300 of proposing an insulin dosage for a diabetes patient according to an exemplary embodiment. FIG. 3 is a workflow diagram for glucose management based on basal/bolus regimen for patients with diabetes type 2.

As indicated by a block 302, the patient is admitted to the hospital. Correspondingly, as indicated by a block 304, a hospital information system is fed with information concerning the patient, in particular age, etc.

In a block 306, it is then decided whether the patient is suffering from diabetes type 2 (T2DM). If this is not the case, see block 308, no further action is taken and the patient is not admitted to the insulin dosage proposal system. If the patient is suffering from diabetes type 2, this patient is enrolled into the glucose management system, see block 310. As indicated by a block 312, the creatinine value is then required as an input. Additionally, see block 314, other parameters such as a bolus insulin type, a basal insulin type, and other parameters are input into the system.

As indicated by a block 345, the therapy of the patient is then initialized. This is performed on the basis of a decision support made for initializing the therapy and using age, weight and creatinine level of the patient as a basis for proposing an initial daily insulin dose, see block 338 and block 340.

As indicated by a block 320, a blood glucose measurement is performed for this patient at different times of the day as a basis for the therapy adjustment, see block 365. The adjustment of the therapy according to block 365 not only considers the blood glucose measurements, but also an iteratively derived new daily insulin dose, compare block 360, using the old daily insulin dose as a basis, see block 362. According to block 347, measured blood glucose values, etc. may be manually input into the system. An insulin administration according to block 355 considers as further input the meal of the patient. Bolus and basal insulin units to be delivered (see reference numeral 350) are provided to the system by a decision support according to a block 364.

When the patient is discharged, see block 368, the reason of discharge is input (see reference numeral 386) and enrolment is stopped according to a block 388.

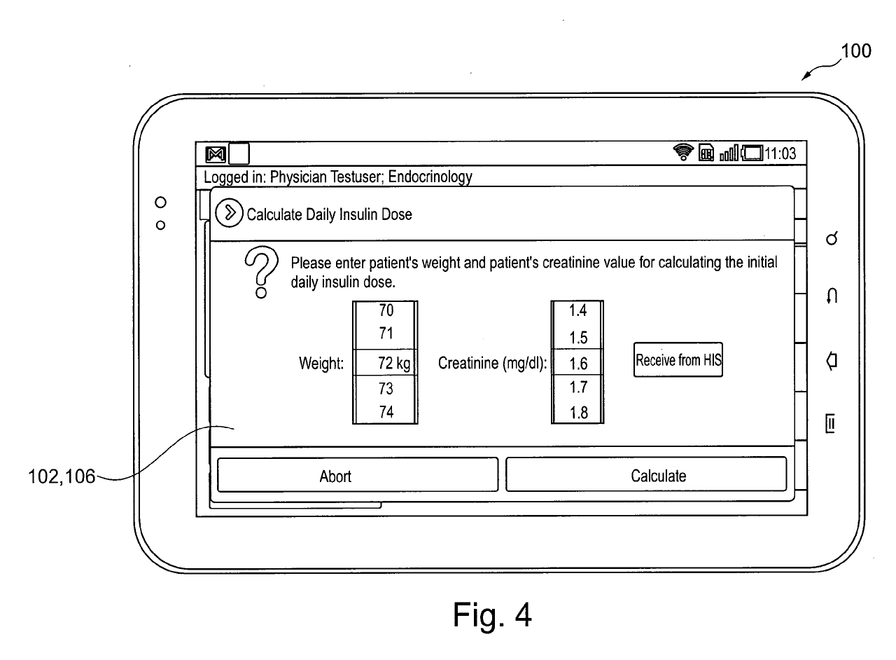

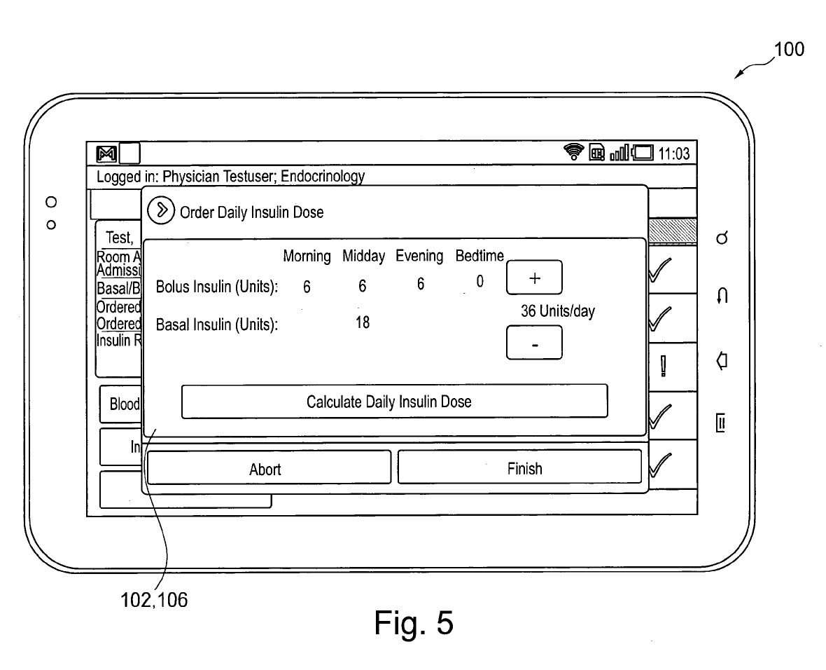

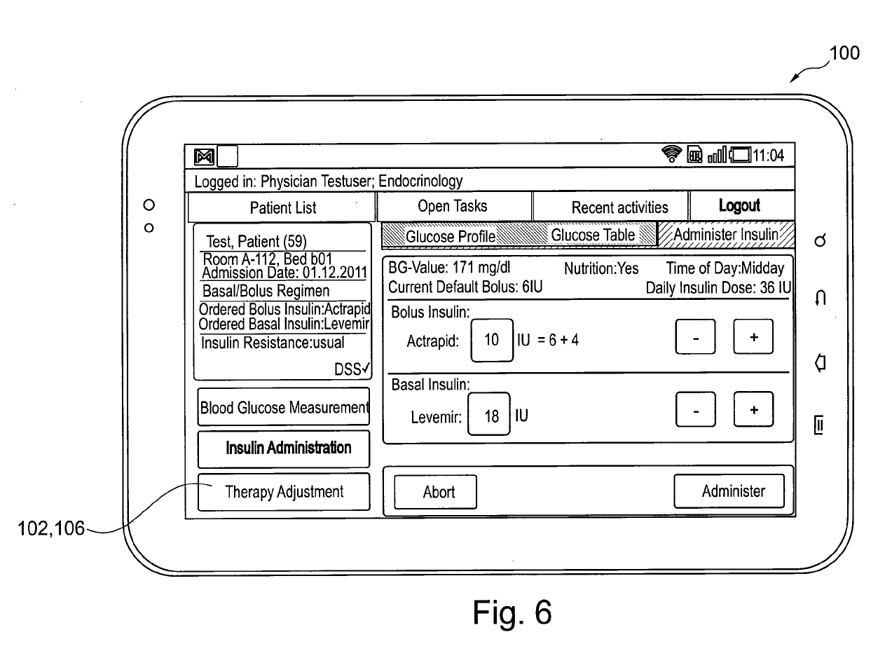

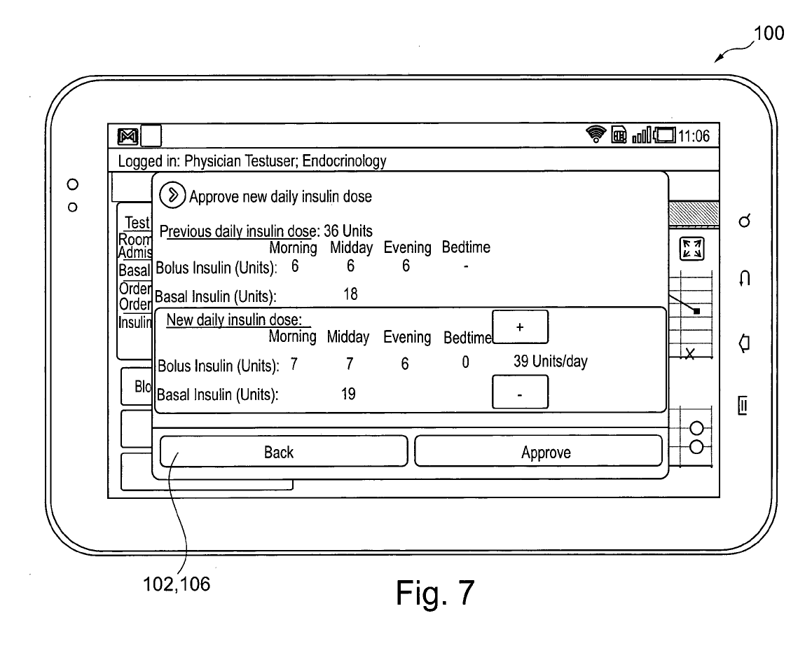

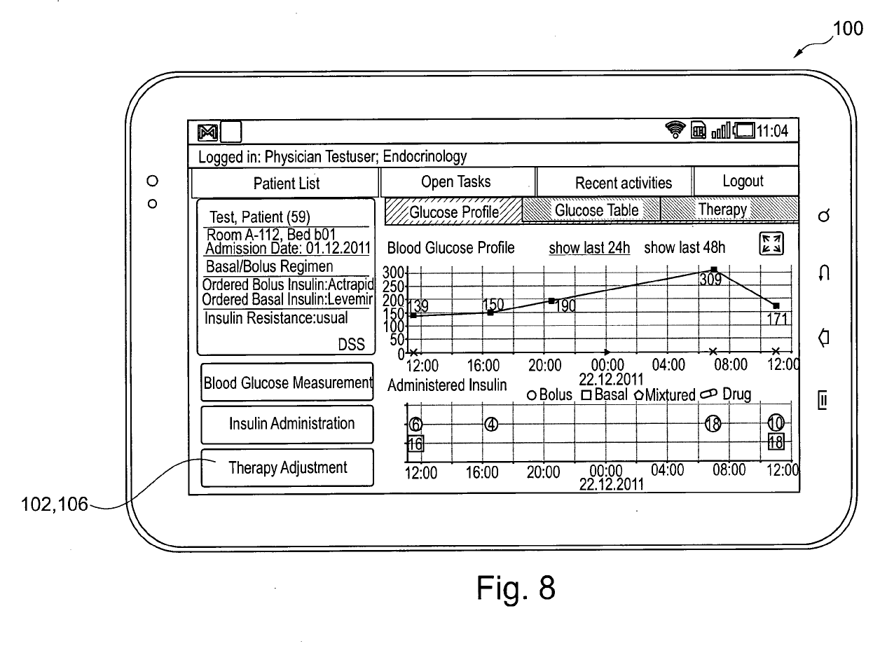

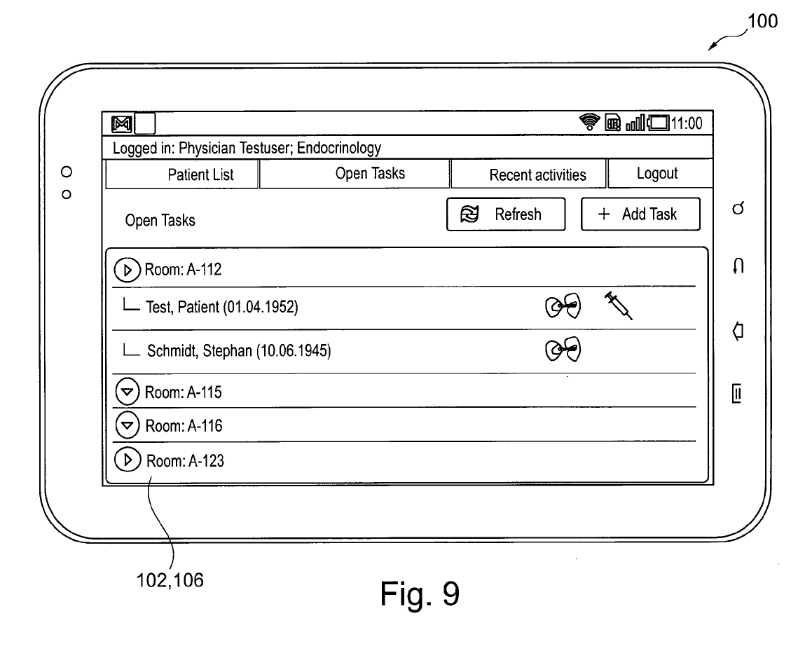

FIG. 4 to FIG. 9 show an embodiment of the device 100 configured as a tablet PC with data displayed on a touchscreen, for proposing an insulin dosage for administration of insulin to a diabetes patient in different operation modes according to an exemplary embodiment of the invention.

Based on the above-described workflow and insulin dosing protocol a tablet-based mobile client/server software application may be implemented in the device 100. The software supports clinical personal directly at the point of care to improve the treatment of patients with diabetes type 2. The screenshots illustrated in FIG. 4 to FIG. 9 show main functions of the software and reproduce the process diagram in FIG. 3 electronically.

FIG. 4 presents input parameters for the initial insulin dose calculation after a patient has been admitted at the ward. FIG. 4 hence shows input parameters for initial calculation of daily insulin dose. Based on the weight and the creatinine value of the patient a daily insulin dose suggestion is provided by the dosing protocol.

FIG. 5 presents the result of the calculation. FIG. 5 hence shows an output of initial daily insulin dose calculation. The system provides the daily insulin dose separated into basal and bolus insulin for the current day.

Additionally, the bolus insulin is divided into doses for the times of the day. A responsible physician can now prescribe the suggestion insulin dose or can change the dose if there are doubts on the suggestion. The type of insulin and other important parameters for the treatment have been entered during the enrolment process.

FIG. 6 shows the insulin dose suggestion for the morning. FIG. 6 hence shows a pre-meal insulin dose suggestion (morning) by the decision support. Bolus insulin is administered before each meal. In the presented screenshot only bolus insulin composed of 6 basis bolus units plus 4 units of supplement bolus are suggested and have to be checked and finally administered. Basal insulin is administered after the ward round at midday (where the therapy adjustment shall be done).

During the ward round, therapy adjustment can be performed by the clinicians each day. The result of the decision support is the suggestion of a new daily insulin dose which has to be approved by a responsible clinician.

FIG. 7 shows the result of the therapy adjustment for a test user. Hence, FIG. 7 shows therapy adjustment to determine a new daily insulin dose.

Then, the results of the blood glucose measurements and the insulin administration are displayed on the main screen of the glucose management system, see FIG. 8. Hence, FIG. 8 shows main functions and provides for a data visualization.

FIG. 9 gives an overview over multiple patients in different hospital rooms all manageable with the same device 100. Furthermore, FIG. 9 illustrates open tasks and therefore reminds a user about actions to be taken.

It should be noted that the term “comprising” does not exclude other elements or steps and the “a” or “an” does not exclude a plurality. Also elements described in association with different embodiments may be combined.

It should also be noted that reference signs in the claims shall not be construed as limiting the scope of the claims.

Implementation of the invention is not limited to the preferred embodiments shown in the figures and described above. Instead, a multiplicity of variants are possible which use the solutions shown and the principle according to the invention even in the case of fundamentally different embodiments.

1. A device for proposing an insulin dosage for a diabetes patient, the device comprising:
an input interface configured for receiving patient glucose level data indicative of glucose level information of the patient;
a processor configured for determining the insulin dosage in an interactive time-dependent manner based on applying the received patient glucose level data to at least only predefined insulin dosage determining criterion; and
an output interface configured for outputting a result of the determining as a proposal for the insulin dosage indicative of doses and assigned times of the day according to which insulin is to be administered to the diabetes patient, wherein the output interface is further configured to display for a user a workflow in accordance to which certain doses of insulin have to be administered to the patient, and is configured to indicate the user other activities, for instance the next glucose measurements to be performed and other events, related to a workflow in a medical facility;
wherein the device is programmed to monitor insulin supply and glucose levels of more than one assigned diabetes patient of the medical facility;
wherein the processor is configured for determining the doses separately for a long-term active insulin and for a short-term active insulin;
wherein the processor is configured for determining insulin delivery doses concerning the short-term active insulin to be administered to the patient in at least three unequal partial doses to be delivered to the patient at different times of the day; and
wherein the processor is configured for determining insulin delivery doses for the short-term active insulin so that each dose comprises a meal-depending base contribution and a patient-depending corrective contribution.

2. The device of claim 1, further comprising at least one feature of the group consisting of:
wherein one of the at least only predefined insulin dosage determining criterion considers historic glucose level data of the patient of the preceding day, including at least one of the group consisting of glucose level data of the patient in the morning and in the evening of the preceding day and glucose level data of the patient averaged over the preceding day;
wherein one of the at least only predefined insulin dosage determining criterion considers one or more historic insulin doses administered to the patient during the preceding day;
wherein one of the at least only predefined insulin dosage determining criterion considers a present amount of insulin in the body of the patient based on previously administered insulin doses and an extrapolation based on the assumption of a linear decrease of administered insulin in the body of the patient.

3.-5. (canceled)

6. The device of claim 1, wherein the processor is configured for determining the dose for the long-term active insulin and the dose for the short-term active insulin with a constant predefined ratio.

7. (canceled)

8. The device of claim 1, further comprising at least one feature of the group consisting of:
wherein the processor is configured for determining the at least three partial doses under consideration of the boundary condition that the largest of the doses of the short-term active insulin is administered to the patient in the morning;
wherein the processor is configured for determining the at least three partial doses under consideration of the boundary condition that a percentage of the short-term active insulin administered in the morning is in a range between 40% and 50%, a percentage of the short-term active insulin administered at midday is in a range between 20% and 30%, and a percentage of the short-term active insulin administered in the evening is in a range between 25% and 35%.

9.-10. (canceled)

11. The device of claim 1, wherein the processor is configured for determining the at least three partial doses of the meal-depending base contribution under consideration of the boundary condition that a percentage of the meal-depending base contribution administered in the morning is in a range between 40% and 50%, a percentage of the meal-depending base contribution administered at midday is in a range between 20% and 30%, and a percentage of the meal-depending base contribution administered in the evening is in a range between 25% and 35%.

12. The device of claim 1, further comprising at least one feature of the group consisting of:
wherein the meal-depending base contribution has always a positive value, preferably with an absolute value larger than an absolute value of the patient-depending corrective contribution;
wherein the patient-depending corrective contribution selectively has a positive value or a negative value.

13. (canceled)

14. The device of claim 1, further comprising at least one feature of the group consisting of:
wherein the processor is configured for determining an daily initial insulin delivery dose to be administered to the patient at the first time of proposing an insulin dosage for this patient based on one or more initial dose determining criteria, including age, weight and creatinine level of the patient;
wherein the processor is configured for determining a subsequent insulin delivery dose to be administered to the patient after administering the initial insulin delivery dose based on the initial insulin delivery dose and based on glucose level data of the patient;
wherein the processor is configured for determining a next day insulin delivery dose to be administered to the patient based on the preceding day insulin delivery dose, and based on glucose level data of the patient at the preceding day and at the current day;
wherein the processor is configured for determining the next day insulin delivery dose based on the glucose level data of the patient in the morning of the present day and in the evening of the preceding day and glucose level data of the patient averaged over the preceding 24 hours;
wherein the processor is configured for determining a future insulin delivery dose of bolus insulin to be administered to the patient under consideration of an estimated amount of insulin being present in the body of the patient;
wherein the processor is configured for reducing a late-administered insulin delivery dose of basil insulin administered to the patient with time delay under consideration of the time delay and configured as a tablet.

15.-20. (canceled)

21. The device of claim 1, wherein the processor is configured for triggering a reminder to be output by the output interface upon identifying a lacking execution of an action required in the framework of the insulin dosage.

22. The device of claim 21, wherein the processor is configured for deactivating the insulin dosage upon identifying lack of data required for a reliable dosage recommendation.

23. The device of claim 1, further comprising at least one feature of the group consisting of:
wherein the processor is configured for, before executing a received user command, verifying a user authorization concerning the user command by determining whether the user command complies with user authorizations defined in a corresponding user profile;
wherein the processor is configured for determining the insulin dosage of the interactive time-dependent manner responsive to a user input via the user interface and/or to one or more insulin administration events and/or to one or more glucose measurements and/or to a present time of the day;
wherein the processor is configured for determining a daily insulin dosage for a respective full day, and wherein the output interface is configured for outputting the proposal for the insulin dosage indicative of multiple doses for the full day which sum up to the determined respective daily insulin dosage;
wherein the processor is further configured for determining a home insulin therapy regimen and an insulin dosage to be administered to a respective diabetes patient after discharging the diabetes patient from the medical facility and following the insulin dosage to be administered to the diabetes patient in the medical facility;
wherein the processor is configured for determining the home insulin dosage with a variable amount and a fixed ratio between a long-term active insulin and a short-term active insulin, whereas the insulin dosage for administration at the medical facility is determined with a variable amount and a variable ratio between the long-term active insulin and the short-term active insulin.

24.-27. (canceled)

28. The device of claim 1,
wherein the processor is configured for determining the proposal for the insulin dosage by:
receiving, in particular via the input interface, at least one parameter indicative of a status of a diabetes patient;
determining or defining a target value of a long-term marker of average glucose concentration of the diabetes patient;
determining a blood glucose target range based on the target value of the long-term marker of average glucose concentration;
receiving, in particular via the input interface, data indicative of a home therapy of the diabetes patient; and
determining a therapy regimen and a proposal for the insulin dosage at the medical facility and/or at a patient's home after discharging the diabetes patient from the medical facility, for the diabetes patient appropriate for achieving the target value and/or the target range.

29. An arrangement for managing insulin delivery to a plurality of diabetes patients of a medical facility, comprising:
a plurality of devices of claim 1, each for proposing an insulin dosage according to which insulin is to be administered to at least a part of the diabetes patients; and
a control entity communicatively coupled to the plurality of devices for communicating device control data and patient-related data between the control entity and the devices.

30. The arrangement of claim 29, comprising at least one feature of the group consisting of:
wherein the devices and the control entity are configured for a wireless communication;
wherein the control entity comprises a patient database storing medical data of the plurality of patients,
wherein each of the plurality of devices is configured for determining the respective insulin dosage under consideration of the medical data of the respective patient,
wherein the control entity comprises a program database storing program code for determining the insulin dosage,
wherein each of the plurality of devices is configured for displaying the insulin dosage determined by executing the program code.

31.-32. (canceled)

33. A method of proposing an insulin dosage for a diabetes patient, the method comprising:
receiving patient glucose level data indicative of glucose level information of the patient;
determining the insulin dosage in an interactive time-dependent manner based on applying the received patient glucose level data to at least only predefined insulin dosage determining criterion;
outputting a result of the determining as a proposal for the insulin dosage indicative of doses and assigned times of the day according to which insulin is to be administered to the diabetes patient, wherein a workflow in accordance to which certain doses of insulin have to be administered to the patient is displayed to a user, and other activities are indicated to the user, for example the next glucose measurements to be performed and other events, related to a workflow in a medical facility;
monitoring insulin supply and glucose levels of more than one assigned diabetes patients of the medical facility;
determining the doses separately for a long-term active insulin and for a short-term active insulin;
determining insulin delivery doses concerning the short-term active insulin to be administered to the patient in at least three unequal partial doses to be delivered to the patient at different times of the day; and
determining insulin delivery doses for the short-term active insulin so that each dose comprises a meal-depending base contribution and a patient-depending corrective contribution.

34. The method of claim 33, wherein the method is applied for diabetes patients of a hospital, a nursing home, or a retirement home.

35. The method of claim 33, wherein the method is applied for a diabetes patient at home.

36. The method of claim 33, wherein discrete doses of insulin are administered discontinuously to the diabetes patient.

37. The method of claim 33, wherein the method further comprises:
receiving at least one parameter indicative of a status of a diabetes patient;
determining or defining a target value of a long-term marker of average glucose concentration of the diabetes patient;
determining a blood glucose target range based on the target value of the long-term marker of average glucose concentration;
receiving data indicative of a home therapy of the diabetes patient;
determining a therapy regimen and a proposal for the insulin dosage at the medical facility and/or at a patient's home after discharging the diabetes patient from the medical facility, for the diabetes patient appropriate for achieving the target value and/or the target range.

38. (canceled)

39. The device of claim 1, wherein the system deactivates to increase patient's safety when data concerning glucose measurement required for a reliable dosage recommendation are missing.

40. The device of claim 1, wherein a morning dose is larger than a midday dose, provided that the long-term active insulin is administered at midday.
